(19) United States
(12) Patent Application Publication
(10) Pub. No.: US 2024/0237916 A1
(43) Pub. Date: Jul. 18, 2024

(54) SYSTEM AND METHOD FOR OPTICAL PRESSURE SENSING OF BODY PART

(71) Applicant: THE GENERAL HOSPITAL CORPORATION, Boston, MA (US)

(72) Inventors: Hamid Ghaednia, Boston, MA (US); Joseph H. Schwab, Boston, MA (US); Sophie Lloyd, Boston, MA (US); Kelsey Detels, Boston, MA (US); Allison Sweeney, Boston, MA (US); David Shin, Boston, MA (US); Amanda Lans, Boston, MA (US)

(21) Appl. No.: 18/558,001

(22) PCT Filed: May 2, 2022
(86) PCT No.: PCT/US2022/027202

§ 371 Date: Oct. 30, 2023

Related U.S. Application Data
(60) Provisional application No. 63/182,736, filed on Apr. 30, 2021.

(51) Int. Cl. A61B 5/103; A61B 5/00; A61B 5/026; A61B 5/22; A61B 90/00; G06T 7/00; G06T 7/90; G06T 11/20

(52) U.S. Cl. CPC A61B 5/1036; A61B 5/0261; A61B 5/225; A61B 5/6895; A61B 5/702; A61B 5/7267; A61B 5/7278; G06T 7/0012; G06T 7/90; G06T 11/206; A61B 2090/064; G06T 2207/10024; G06T 2207/10152; G06T 2207/30104

(57) ABSTRACT

A system for analyzing a body part of a subject includes a sensor interface having a surface for receiving the body part. A light source emits light having multiple wavelengths within the sensor interface to be reflected by the body part interacting with the surface. An imaging system captures images of the reflected light. A computing device generates a pressure distribution map of the body part on the surface based on the images.

### RELATED APPLICATIONS

This application claims the benefit of U.S. Provisional Application Ser. No. 63/182,736, filed Apr. 30, 2021, the entirety of which is incorporated by reference herein.

### TECHNICAL FIELD

The present invention relates generally to sensor systems, and specifically to an optical pressure sensing system for analyzing body parts.

### BACKGROUND

The pattern in which we apply force and pressure to our hands and feet can reveal a lot about how our musculoskeletal and nervous systems behave. Current pressure panels use electrical sensors to monitor pressure distribution on a subject's feet. However, due to their limited resolution, these devices fail to provide sufficiently accurate data. An accurate pressure distribution pattern of the feet can aid our understanding of different orthopedic complexities. Additionally, such a pattern has the potential to predict orthopedic complexities, help with the development of preventative and therapeutic mechanisms, and assess progress following musculoskeletal injuries and surgeries. An example of such an application is the development of quantitative assessment of recovery for patients with lumbar stenosis, cervical myelopathy, arthroplasty patients, ankle fracture, nervous system complexities, etc.

The potential of connecting light intensity to pressure distribution of feet using a glass panel illuminated by light strips was first realized by Betts and Duckworth (1978). The method was further investigated and improved by Duckworth (1982). However, until recently, there wasn't an equation that described the contact dynamics. Sharp (2018) at the University of Nottingham tested a device and generated a third order equation relating light intensity and pressure. This equation, however, does not capture spatial differences in material and optical properties of a human foot and therefore is not useful for calculating real force distribution under feet, which could be useful for patient health assessment.

### SUMMARY

In one example, a system for analyzing a body part of a subject includes a sensor interface having a surface for receiving the body part. A light source emits light having multiple wavelengths within the sensor interface to be reflected by the body part interacting with the surface. An imaging system captures images of the reflected light. A computing device generates a pressure distribution map of the body part on the surface based on the images.

In another example, a method for analyzing a body part of a subject includes receiving the body part on a contact surface of a sensing interface. Light having multiple wavelengths is emitted within the sensing interface. Images of the light reflected by the body part interacting with the contact surface are acquired. A pressure distribution map of the body part on the contact surface is generated based on the images.

In another aspect, taken along or in combination with any other aspect, the light source emits a combination of red, blue and green light.

In another aspect, taken along or in combination with any other aspect, force sensors measure force applied by the body part to the surface.

In another aspect, taken along or in combination with any other aspect, the computing device generates the pressure distribution map based on the images and the measured force.

In another aspect, taken along or in combination with any other aspect, the pressure distribution map plots force versus contact area for the body part across the surface.

In another aspect, taken along or in combination with any other aspect, the surface is planar.

In another aspect, taken along or in combination with any other aspect, the surface is spherical.

In another aspect, taken along or in combination with any other aspect, the surface is cylindrical.

In another aspect, taken along or in combination with any other aspect, the surface is curved.

In another aspect, taken along or in combination with any other aspect, the computing device is configured to generate a spatial mechanical properties map of the body part based on the images.

In another aspect, taken along or in combination with any other aspect, the images include a distribution of light intensity and different light colors.

In another aspect, taken along or in combination with any other aspect, at least one factor from at least one of the images is extracted. The at least one factor is input into a trained machine learning algorithm to generate the pressure distribution map.

In another aspect, taken along or in combination with any other aspect, the body part is three-dimensionally analyzed as a viscoelastic material based on the images. The pressure distribution map is generated based on the analytical model.

In another aspect, taken along or in combination with any other aspect, a perfusion map is generated to assess blood circulation based on the spatial mechanical properties map.

In another aspect, taken along or in combination with any other aspect, blood circulation in the body part is assessed based on a distribution of different colored light in the reflected light images.

In another aspect, taken along or in combination with any other aspect, the temperature of the body part is varied and the blood circulation of the body part at the different temperatures assessed based on changed in the colored light distribution in the light images.

In another aspect, taken along or in combination with any other aspect, a first pressure distribution map is generated before a treatment of the body part begins and a second pressure distribution map is generated after the treatment of the body part begins.

Other objects and advantages and a fuller understanding of the invention will be had from the following detailed description and the accompanying drawings.

### BRIEF DESCRIPTION OF THE DRAWINGS

FIG. 1A is a schematic illustration of an example body part analyzing system in accordance with the present invention.

FIG. 1B is a section view of a sensor interface for the system taken along line 1B-1B of FIG. 1A.

FIG. 2A is a schematic illustration of an alternative sensor interface.

FIG. 2B is a section view taken along line 2B-2B of FIG. 2A.

FIG. 3A is a schematic illustration of another alternative sensor interface.

FIG. 3B is a section view taken along line 3B-3B of FIG. 3A.

FIG. 4 is a block diagram of the body part analyzing system.

FIG. 5A is an example neural network for use with the system.

FIG. 5B is an example node of the neural network.

FIG. 6 is a schematic illustration of the system being used to generate a real pressure distribution map based on a light reflection map of the feet of an individual.

FIG. 7 is a graph illustrating loading and unloading phases of a foot on a sensor interface.

FIG. 8 is a force distribution map of a foot on a sensor interface.

### DETAILED DESCRIPTION

The present invention relates generally to sensor systems, and specifically to an optical pressure sensing system for analyzing body parts. To this end, the system is configured to capture images of multiple wavelength light reflected off an individual's body part(s) while engaging a sensor interface. Data extracted from the reflected light images can be transformed or converted into a real time pressure distribution map for visualizing the same and making diagnoses/assessments therefrom.

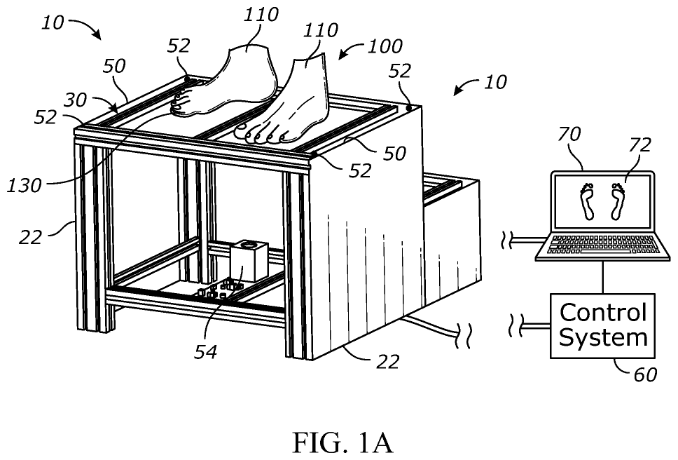

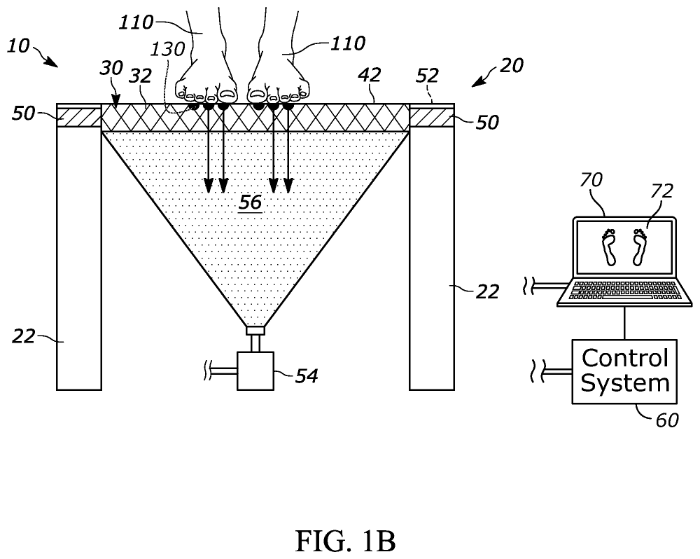

FIGS. 1A-1B illustrate an example body part analyzing system 10 in accordance with the present invention. In one instance, the system 10 is configured as a frustrated total internal reflection (“FTIR”) system. A frustrated internal reflection occurs when light travels from a material with a higher refractive index in the direction of a lower refractive index at an angle greater than its critical angle. It becomes “frustrated” when a third object comes into contact with the surface and alters the way the waves propagate, and the capture of which may be used to produce a surface pattern. FTIR is able to detect the interface/contact area at a very high resolution through image processing. Through software, measured light intensity can be sorted into a gradient of high-to-low intensity pixels.

With this in mind, the system 10 includes a platform or frame 20 having a series of legs 22 supporting a sensor interface 30. The sensor interface 30 includes a panel 32 having a contact surface 42 facing upwards (as shown). In this example, the contact surface 42 is planar. The panel 32 is made from a light transmissive material, such as glass, polycarbonate, acrylic or other transparent medium. Alternatively, the panel 32 could be formed from a colored medium.

A light source 50 is provided for emitting light through the cross-section of the panel 32. In other words, the light is emitted between the surfaces defining the thickness of the panel 32. The light source 50 can be formed as a series of light emitting diodes (LEDs) arranged in a predefined pattern. The light source 50 can emit light from one or more wavelengths. In one example, the light source emits red, green, and blue light (RGB). Alternatively, the light source emits ultraviolet and/or infrared red. As shown, the light source 50 is provided on opposite sides of the frame 20 such that the LEDs all emit light towards the center of the panel 32. Alternative configurations for the light source 50 are also contemplated.

Pressure sensors 52 are provided on the sensor interface 30 and generate signals indicative of the location and value of pressure exerted on the contact surface 42. The pressure sensors 52 can, however, be omitted.

An imaging system 54 is positioned within the frame 20 and includes one or more cameras. As shown, the imaging system comprises a single camera 54 having a field of view 56 facing upwards (as shown) towards the sensor interface 30 for capturing images of/around the contact surface 42. A projection material (not shown) can be applied to the underside of the panel 32 for displaying an image to be captured and scattering light.

A controller or control system 60 is connected to the light source 50, pressure sensors 52, and camera 54. The controller 60 is configured to control the light source 50, e.g., control the color(s), intensity and/or duration of the light emitted by the light source. The controller 60 is also configured to receive signals from the camera 54 indicative of the images taken thereby and signals from the pressure sensors 52 indicative of the pressure exerted on the contact surface 42. A computer or computing device 70 having a display 72 is connected to the control system 60. The computing device 70 can be, for example, a desktop computer, smart phone, tablet, etc.

The sensor interface 30 is configured cooperate with the light source 50, pressure sensors 52, camera 54, and controller 60 to analyze interactions between a subject/individual 100 and the sensor interface. The subject 100 can by any mammal, including a human or animal (dog, cat, horse, etc.) and, thus, the sensor interface 30 can be configured to function in both a hospital/clinical setting as well as in veterinary applications. That said, the subject 100 shown is a human individual.

In the example shown in FIGS. 1A-1B, feet 110 of the individual 100 interact with the contact surface 42 of the sensor interface 30. That said, and depending on the size/scale of the contact surface 42, the individual 100 may step, walk, run, jump on, stand, etc., on the sensor interface 30. Regardless, the feet 110 engage the contact surface 42 over an interface zone 130.

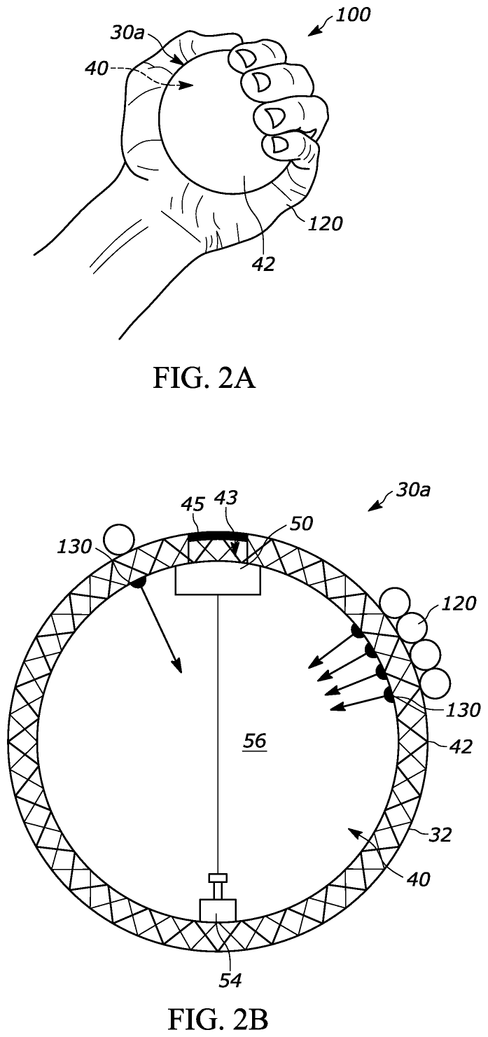

FIGS. 2A-2B illustrate an alternative configuration for the sensor interface, indicated at 30a for clarity. In this example, the sensor interface 30a is spherical. With this in mind, the sensor interface 30a includes a spherical panel 32 defining a cavity 40. An annular opening 43 is formed in the panel 32 and extends from outside the sensor interface 30a and radially inward to the cavity 40. The light source 50 is provided in the opening 43 and configured to emit light circumferentially through the material cross-section of the panel 32. In other words, the light is reflected between the surfaces defining the radial thickness of the panel 32. The sensor interface 30a can include additional openings 43 and corresponding light sources 50 provided therein for helping emit light through the entire cross-section of the spherical panel 32. The area around the opening(s) 43 is covered with a dark material/paint to ensure FTIR.

The imaging system in this example consists of a single camera 54 provided within the cavity 40 and having a field of view 56 substantially covering the entirety of the cavity. The camera 54 can be a fish-eye camera with a wide field of view 56. The camera 54 captures images of the frustrated internal reflection as a hand 120 of the individual 100 interacts with the contact surface 42 of the sensor interface 30a. A wireless device (not shown) can help to transmit data between the camera 54 and the computing device 70. The sensor interface 30a may or may not include pressure sensors 52.

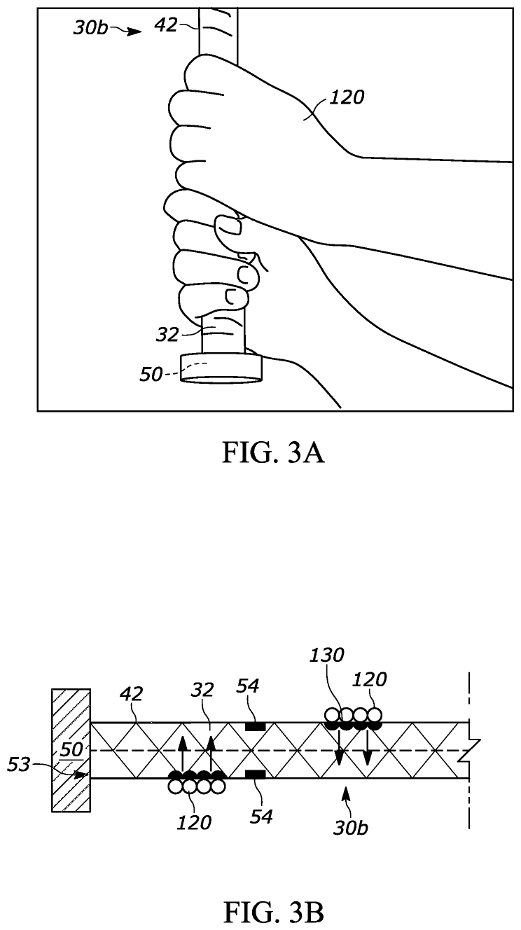

FIGS. 3A-3B illustrate an alternative configuration for the sensor interface, indicated at 30b. In this example, the sensor interface 30b is cylindrical. With this in mind, the sensor interface 30b includes a cylindrical panel 32 that can be solid in cross-section (as shown) or tubular (not shown). The light source 50 is secured to the panel 32 so as to emit light through the material cross-section thereof in a direction extending along/parallel to the longitudinal axis of the sensor interface 30b.

The imaging system in this example consists of a pair of cameras 54 facing each other and secured to opposing sides of the panel 32. Collectively, the fields of view 56 of the cameras 54 substantially cover the entirety of cross-section of the panel 32 where hands 120 of the individual 100 could reasonably be positioned. To this end, each camera 54 can be a fish-eye camera with a wide field of view 56. The camera 54 captures images of the frustrated internal reflection as the hands 120 of the individual 100 interact with the contact surface 42 of the sensor interface 30a. A wireless device (not shown) can help to transmit data between the cameras 54 and the computing device 70. The sensor interface 30b may or may not include pressure sensors 52.

From the above, it is clear that the sensor interface 30, 30a, 30b can assume a wide range of shapes and sizes and, thus, the sensor interface can be configured as a multitude of everyday objects and/or sports equipment that an individual would interact with. This includes, but is not limited to, curved objects such as a football, baseball, soccer ball, golf club, and stairway railing.

Regardless of the configuration of the sensor interface, the imaging system 54 of the system 10 is configured to capture images of multiple wavelength light reflected off the individual's 100 body part(s) while engaging the sensor interface 30, 30a, 30b. The computing device 70 can then extract data from the reflected light images and—either analytically or with machine learning—transform the light intensity data into a real time pressure distribution map for visualizing the same and making diagnoses/assessments therefrom.

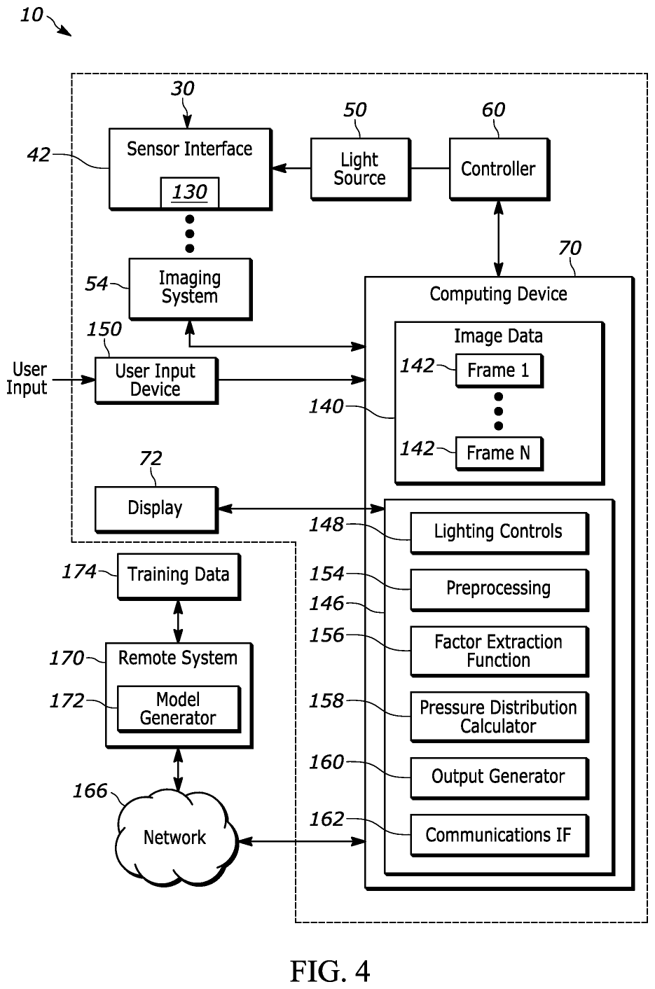

With this in mind, FIG. 4 depicts a more detailed breakdown of exemplary components of the system 10. The imaging system 54 is configured to record images of the interface zone 130 during a test time interval to provide corresponding image data 140. The image data 140 includes a plurality of image frames 142 shown as frames 1−N, where N is a positive integer denoting the number of image frames. The number of frames N can vary depending upon the length of the test interval and the frame rate at which the imaging system 54 records the image frames.

The image data 140 can be stored in one or more non-transitory machine-readable media, i.e., memory, of the computing device 70. The memory can include volatile and non-volatile memory, and may be local to the system, external to the system or be distributed memory. The image data 140 can also be stored in the imaging system 54 and then transferred to corresponding memory of the computing device 70 during or after the test interval has completed.

Since the light source 50 emits multiple wavelength light into the sensor interface 30, the image data 140 includes not only different light intensity but also different light color. It will be appreciated that human body parts are not homogenous and therefore different portions of, for example, the foot can exhibit different surface roughness, Young's Modulus, elasticity, etc. Different wavelength light reacts differently to these different material properties and, thus, imaging body parts with multiple wavelength light enables the system 10 to spatially map the material properties of the body part.

The imaging system 54 provides image data 140 that includes a plurality of image frames 142 based on a frame rate at which the frames are acquired by the imaging system. The frame rate may be fixed or it may be variable. In one example, the frame rate may be one frame every ten seconds or faster, such as one frame every seven seconds, one frame every 4 seconds, one frame every two seconds, one frame per second or up to a rate of full motion video, e.g., up to about 100,000 frames per second. The number of frames determines the resolution of the interface zone 130 contained in the image data 140.

The computing device 70 can be programmed with instructions 146 executable by one or more processors of the computing device. The instructions 146 include lighting controls 148 programmed to provide instructions to the controller 60 for controlling operating parameters of the light source 50. For example, a user input device 150 can be coupled to or part of the computing device 70 and, in response to a user input via the user input device, can operate the lighting controls 148 to change the color, intensity, and/or duration of the light entering the panel 32 of the sensor interface 30.

As an example, the user input device 150 can be implemented as a keyboard, mouse, touch screen interface, or a remote user device, e.g., a cellphone or tablet connected to the computing device 70 through an interface to enable user interaction with the computing device as well as the system 10, more generally.

The computing device 70 can be coupled to the sensor interface 30 and controller 60 by a physical connection, e.g., electrically conductive wire, optical fibers, or by a wireless connection, e.g., WiFi, Bluetooth, near field communication, or the like. In one example, the controller 60 and computing device 70 can be external to the sensor interface 30 and be coupled therewith via a physical and/or wireless link. Alternatively, the controller 60 and computing device 70 can be integrated into the sensor interface 30 (not shown).

The instructions 146 are also configured to analyze the multiple wavelength light reflected across the interface zone 130 based on the image data 140 such as to re-characterize or transform the reflected light data into as a real pressure distribution map. The computing device 70 can also be configured to determine a condition, e.g., a diagnosis or prediction, of the individual 100 based on the image data 140.

The instructions 146 include a preprocessing function 154 to process the image data 140 into a form to facilitate processing and analysis. To this end, the preprocessing function 154 can also include a pixel scaling function programmed to scale pixels in each of the respective image frames 142 so that pixel values within each respective image frame are normalized. Once the image data 140 is processed, a factor extraction function 156 extracts one or more factors from the image data 140.

The factor can be any particular data extracted from one or more image frames 142, such as body part contact area, colored pixel distribution, section of the foot, loading or unloading phase of the interaction, and/or first and second moment of contact area. More specifically, the inputs I can include, but are not limited to: the distribution of red, green, and blue pixels within the images as well as their values; the contact areas between each/both legs or hands and the sensor interface; the center of contact between the body part and the sensor interface; the reaction forces on each/both legs or hands from the sensor interface; pressure distribution values across the sensor interface based on all the light, only the red pixel values, only the blue pixel values, etc.; the center of pressure on each/both feet or hands on the sensor interface; the first, second, third, and fourth moment of the center of pressure on the sensor interface; and/or the Fast Fourier transform of the contact area in the time dimension.

Fast Fourier Transforms (FFT) can be performed on the contact area in time dimension, the pressure and force in the time dimension, the center of the contact area in time dimension, and the center of pressure and center of force in time dimension. The integral of the FFTs of the pressure and force, the contact area and force, the center of pressure and center of force, and the center of contact area can also be performed. Additionally, standard deviations of the centers of area, pressure, and force as well as of the contact area, pressure, and force can be obtained.

The instructions 146 also include a pressure distribution calculator 158. The pressure distribution calculator 158 is programed to analyze the determined pixel values for some or all of the respective image frames 142 and estimate properties of the interface zone 130 based on such analysis. More specifically, the pressure distribution calculator 158 is configured to analyze the image data 142 and estimate the pressure distribution across the interface zone 130.

In one example, the pressure distribution calculator 158 is programmed to analytically determine the pressure distribution across the interface zone 130. To this end, the test data can be combined with 3D modeling analysis in order to generate analytical solutions that map light intensity to real pressure distribution. In this approach, the foot 110 is modeled on a finite element environment where mechanical properties of the foot are first measured by real subjects. The model is then subjected to different static and dynamic load scenario. The results of this numerical analysis is then used for fitting mathematical models on the numerical data.

In our experiments, we observed that the foot 110 behaves mostly similar to a viscoelastic material or even metal over elastic materials. That said, the finite element analysis was performed while treating the foot as a viscoelastic material and not an elastic material. The pressure distribution calculator 158 can thereby develop the analytical model based on the numerical analysis.

In another example, the pressure distribution calculator 158 includes a machine learning model trained to recognize patterns in data derived or extracted from the image frames 142 in order to predict the pressure distribution of the body part as it interacts with the sensor interface 30. The machine learning model implemented by the pressure distribution calculator 158 can utilize one or more types of models, including support vector machines, regression models, self-organized maps, k-nearest neighbor classification or regression, fuzzy logic systems, data fusion processes, boosting and bagging methods, rule-based systems, artificial neural networks (ANN) or convolutional neural networks. In one example, the machine learning is an ANN having supervised or semi-supervised learning algorithms programmed to perform pattern recognition and regression analysis to predict a pressure distribution across the sensor interface 30 based on reflected light images.

In particular, the ANN can be a trained, deep machine learning model having a neural network with a plurality of layers trained to analyze some or all of the respective image frames 142 to perform corresponding pattern recognition and regression analysis to correlate detected light intensity with pressure distribution. The ANN (or other function implemented by the pressure distribution calculator 158) can also diagnose an orthopedic disorder based on the pressure distribution map.

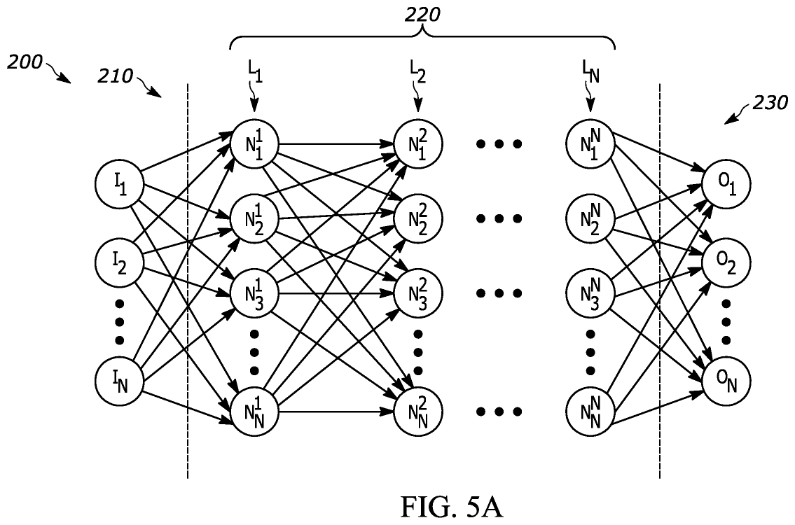

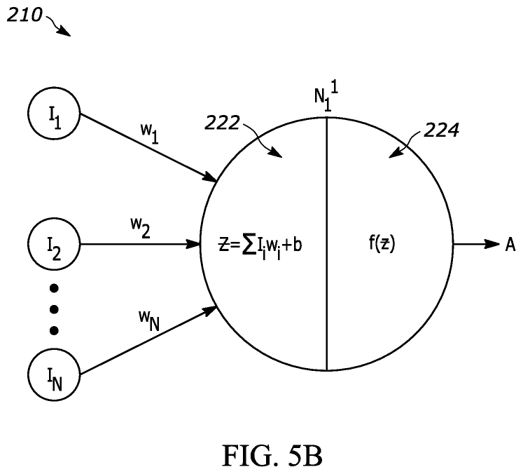

One example machine learning neural network 200 integrated into the pressure distribution calculator 158 is shown generally in FIG. 5A. The neural network 200 includes an input layer 210, one or more hidden layers 220, and an output layer 230. In one example, multiple hidden layers—indicated at L₁, L₂, . . . L_{N}—can be used and, thus, the neural network can use deep learning. The neural network 200 can be configured as a fully connected, feedforward neural network. With that said, each input in the input layer 210 is indicated at I₁, I₂, . . . Iₙ. Each output in the output layer 230 is indicated at O₁, O₂, . . . O_{N}. Each hidden layer 220 is formed from a series of nodes N₁, N₂, . . . N_{N}.

The inputs I delivered/entered into the input layer 210 are obtained from the factor extraction function 156 in the computing device 70 and used to help train the neural network 200. In other words, the test data (or at least a portion thereof) obtained from the captured images and pressure sensor signals is used as training data in order to perfect the machine learning algorithms. It will be appreciated that the inputs I can also include data from one or more of the pressure sensors 52 (when present). That said, each input I is assigned a different weight w preselected by the user or randomly selected.

Referring to FIG. 5B, the neuron N determines a sum z of the products of the inputs I and their associated weights w fed to the neuron, plus a bias b (which can also be preselected or randomly selected). This sum function is indicated at 222 and is the same for all the neurons N in the first hidden layer L₁. The bias b can be the same for each sum function 222 in the first hidden layer L₁.

An activation function 224 converts the result of the summing function 222 into an output A for that particular neuron N. The activation function 224 can be, for example, a binary step function, linear activation function or non-linear activation function. Example non-linear activation functions include, but are not limited to, sigmoid/logistic, Tan-h function, Rectified Linear Unit, Leaky Rectified Linear Unit, Parametric Rectified Linear Unit, and Exponential Linear Unit. Other activation functions known in the art are also contemplated.

The first hidden layer L₁ uses the input values I, weights w, and bias b to calculate an output A, which is sent to each node N in the second hidden layer L₂. The output A of each node N in the first layer L₁ also has a predetermined weight w and, thus, the summing and activation function calculations are performed for each node N in the second hidden layer L₂. The process is repeated for each successive hidden layer N until the final hidden layer Nₙ calculates and exports the predicted output(s) O. Each output O can be a single value, e.g., a pressure value at a particular location on the contact surface 42, or array/matrix of values.

It will be appreciated that the bias b is the same for each neuron N in a particular hidden layer L. The bias b can be different, however, for different hidden layers, e.g., the second hidden layer L₂ can have a different bias b in its summing function than the bias b in the first hidden layer L₁. Additionally, the activation function can be the same or different for each neuron N within any particular hidden layer L.

Regardless, the prediction and outputs O from the test data are assessed for accuracy and the algorithm re-run with new values for weights w and biases b chosen. The cycle is repeated until a threshold correlation between inputs I and outputs O is achieved. It will be appreciated that the activation function, number of hidden layers, number of outputs O and/or type of outputs could also be changed between learning cycles until an appropriate combination is found.

Once the neural network 200 is properly trained, it can be used to generate predicted outputs O when new/live data in used at the inputs I. In other words, once test data from healthy patients is used to train and validate the algorithm, data can be extracted from new imaging system 54 images and supplied at the inputs I to generate predicted outputs O that in this case relates light intensity values to real pressure distribution.

Returning to FIG. 4, the instructions 146 implemented by one or more processors of the computing device 70 can also include an output generator 160. The output generator 160 is programmed to generate the output data that can be provided to the display 72 or other output device to provide a tangible representation of the pressure distribution map determined by the pressure distribution calculator 158. This can include a textual and/or graphical representation of the real-time pressure distribution of the foot 110 at the interface zone 130.

The computing device 70 can also include a communications interface 162 to communicate through one or more networks 166, such as for communications with a remote system 170. The communication interface 162 can be implemented to communicate with the network 166 using one or more physical connections, e.g., an electrically conductive connection or optical fiber, one or more wireless links, e.g., implemented according to an 802.11x standard or other short-range wireless communication, or a network infrastructure that includes one or more physical and/or wireless communications links.

The remote system 170 can include a server, a general purpose computing device, e.g., notebook computer, laptop, desktop computer, workstation, smartphone or the like, and/or it can be a special purpose system configured to interact with one or more of the systems 10 via the network 166. In another example, the remote system 170 may send program instructions to the computing device 70 to configure and/or update its operating program instructions 146. The remote system 170 can include a model generator 172 that is programmed to execute instructions for generating one or more machine learning models that can be provided to the pressure distribution calculator 158 through the network 166.

The remote system 170 can also supply the machine learning model in the pressure distribution calculator 158 with additional training data 174, which can be previously acquired test data captured by the imaging system 54 and/or other data acquired elsewhere. The training data 174 can therefore supplement or be used in lieu of the test data acquired by the imaging system 54. The training data 174 can also be used to validate the neural network 200 once the test data teaches the network.

Returning to FIGS. 1A-1B, when the system 10 is operating, the light source 50 emits multiple wavelength light into the cross-section of the panel 32. This light is trapped within the panel 32 and travels therethrough as denoted generally by the representative light lines. When the individual 100, or an article worn by the individual, such as a shoe (not shown), makes contact with the contact surface 42, the total internal reflection is frustrated at the interface zone 130 and directed generally towards the camera 54.

The camera 54 images this reflection and sends the image data 140 to the computing device 70, which can generate a composite light reflection map across the entire contact surface 42. In other words, the computing device 70 receives image signals from the camera 54, interprets/analyzes those signals, and generates a composite map illustrating the reflection of light off the foot 110 and along the contact surface 42. Since multiple wavelengths of light are emitted by the light source 50, the composite map illustrates how multiple wavelengths of light, e.g., RGB light, interacts with the foot 110 on the contact surface 42.

At the same time, the pressure sensors 52 send signals to the computing device 70 indicative of the pressure exhibited by each foot 110 on the contact surface 42. The individual 100 can perform multiple interactions with the sensor interface 30 such that multiple data sets are compiled. The interactions can be repeating the same motion, e.g., standing on one foot 110, standing on the other foot, jumping, stepping on and/or off the contact surface 42, etc., or performing multiple, different motions. Additionally, multiple individuals 100 can have one or more interactions with the sensor interface 30 in order to compile a large set of test data including both light intensity data and (if present) pressure sensor data.

Using the analytical approach, the pressure distribution calculator 158 relies on the image data 140 to create the spatial mechanical property map of the feet 110. The data therein is then used in three-dimensional, finite element analysis of the foot 110, which is subjected to different static and dynamic load scenarios. Based on this analysis, an analytical model can be developed by the pressure distribution calculator 158 to correlate light intensity within the images to pressure distribution.

Using the machine learning approach, after the neural network 200 is trained using the test data, a new individual 100 stands on the sensor interface 30 and the image/sensor data collected as previously described. The factor extraction 156 extracts at least one of the aforementioned factors from each image frame 142 and uses them as the inputs I for the trained neural network 200. The trained neural network 200 (though the pressure distribution calculator 158) then relies on the extracted factors to generate the outputs O. The output generator 160 then sends the desired output, e.g., a predicted pressure distribution map of the foot 110, to the display 72 to allow the individual 100 and accompanying physician/medical professional to view a live feed of the pressure distribution as the individual interacts with the sensor interface 30.

The system 10 of the present invention is thereby capable of more accurately accounting for variations in the geometry and composition of the body part interacting with the sensor interface 30 in order to translate light intensity images into real pressure distribution in real-time. It will be appreciated that the same or similar methodologies can be used whether the sensor interface is planar, curved, spherical or cylindrical and/or whether the body part is a foot, hand, etc.

The system 10 of the present invention advantageously uses empirical formulation on non-dimensionalized (normalized) parameters to account for differences in material properties exhibited at different portions of the same body part. For instance, the loading and unloading phases of the foot on the sensor interface 30 can be separated and an empirical formulation for each phase developed. In this instance, the initial condition of the unloading phase is based on the end of the loading phase. The empirical formulation models are based on actual experimental data of healthy subjects.

The system 10 allows for not only a predicted diagnosis for the individual but also an assessment of recovery from an injury or condition. To this end, the neural network 200 can output health predictions based on input data I from the individual extracted from the images. The display 72 can receive the outputs O from the computing device 70 and display possible diagnoses and/or a recovery progress report that both the individual 100 and medical professional and inspect and assess.

For instance, the system 10 can be used to assess if/to what extend balance, pronation or supination have improved following a foot injury or map hand/grip pressure on a ball following a hand injury. The system 10 can therefore output/display specific data related to such assessments, including balance measures, weight bearing ratios, etc. Moreover, since the neural network 200 is already trained the medical professional is able to view the interaction between the individual 100 and the sensor interface 30 in real-time while different tests, e.g., Romberg test, are performed.

### Blood Circulation

The system 10 can be configured to map and monitor blood circulation in a body part. Blood circulation/perfusion affects the optical and mechanical properties of the foot 110 and, thus, using multiple wavelengths of light within the sensing interface 30 advantageously allows for a more detailed depiction and analysis of the foot. For example, rubbing the arch of a subject's foot 110 causes the pressure images to appear redder. This allows for quantitative analysis of blood circulation in a patient's feet 110.

To this end, RGB value distribution over the contact area is one of the factors analyzed for understanding the effects of blood circulation. Differences in the RGB distribution when the temperature of the subject's feet 110 is unchanged are first determined (this can be the test data). Once the test data is analytically modeled or used to teach a machine algorithm, the extent to which temperature or blood circulation changes the RGB distribution, the recovery time for feet to return to a baseline in response to temperature fluctuations, and/or what area of the foot experiences the most change can be analyzed. These and other factors can be assessed by generating one or more perfusion maps based on the RGB value distribution(s) over time.

The changes in the mechanical properties captured in the contact area can also assessed. In particular, blood circulation changes the contact area of the foot 110 by changing its mechanical properties. We observed up 70% change in the contact area after the foot 110 was exposed to heat. The blood circulation also changes the force distribution by changing the spatial mechanical properties of the foot. That said, how blood circulation changes the pressure distribution of different sections of the foot can also be studied.

A comparison between each of the same individual's feet 110 can also be studied. To this end, two-dimensional cross correlation methods, such a 2D FFT correlation method, can be performed on the mirrored image of one foot and the original image of the contralateral to match the contact areas while the subject is standing on one foot or both feet simultaneously. The mechanical properties, contact area patterns, and pressure distribution patterns can then be compared between the two feet.

### Orthopedic Conditions

The system shown and described herein can be used to identify and assess a wide range of orthopedic conditions, and can be used to help track treatment plans seeking to ameliorate said conditions. For instance, the system can be used to detect injury to a patient's ACL and/or help to assess recovery progress therefrom. The force distribution map created by the patient standing on the sensor interface can help identify and quantify any balance issues that might otherwise be imperceptible to the treating physician. Similar balance issues can be identified when the patient has an injury to the foot, ankle, knee, hand, etc.

### Clinical Applications

There are several clinical applications for this work. Certain embodiments and application of the present invention can include, but are not limited to, nervous system activity in that accurate pressure distribution measurements can quantify balance for patients with nervous system or musculoskeletal complexities such as relapsing Multiple Sclerosis (MS). Low back pain, joint instability, and stroke can also be assessed by measuring factors such as center of pressure, center of area, and noise analysis of the contact area, force and pressure distribution that quantify balance.

Moreover, we can quantify the muscle contraction level through pressure distribution maps. An example of such an embodiment of the proposed device is a flat or curved (open or closed) surface that measures hand or foot pressure simultaneously with effects of blood circulation in order to measure nervous system activity. Another example embodiment of the device is an optical pressure plate in the shape of a piano that patients can be asked to play on while the device is analyzing the pressure distribution and blood circulation of the fingertips.

The system of the present invention can also be used to measure load bearing capacity after surgery, walking patterns, blood circulation, comparison of the pressure distribution and blood circulation between feet. An embodiment of the proposed device for such an application is an appropriately sized pressure plate a patient can walk on or press his/her hand against before and during the treatment to create quantitative assessment of the health condition for patients with musculoskeletal complexities, diabetes, or both. Another embodiment could be an optical pressure plate in the form of stairs and railing where both pressures of the hand and foot are being analyzed. The quantification of current patient functional outcomes and creating new functional outcome score by creating comprehensive quantitative assessment of musculoskeletal patients and improving the design and selection of orthotics are also contemplated.

### Non-Clinical Applications

There are also several non-clinical applications for our device, including sport accessories such as a basketball, baseball, football or soccer ball. This can be used to assess a players' kick, grips, throws etc. and/or to develop commercial balls for comparing amateur to professional players. An example embodiment of the invention includes but is not limited to a transparent baseball that has mechanical properties similar to a real ball that can analyze the pressure distribution of a player's grip, for example, analyzing the grip of a baseball player while throwing the ball.

Furthermore, the device and method described herein can be scaled for scanning interfaces between objects in the nano-scale. In particular, a three dimensional map of the interface can be created by changing the wavelength of the light emitted by the light source(s) from small to large.

### Example

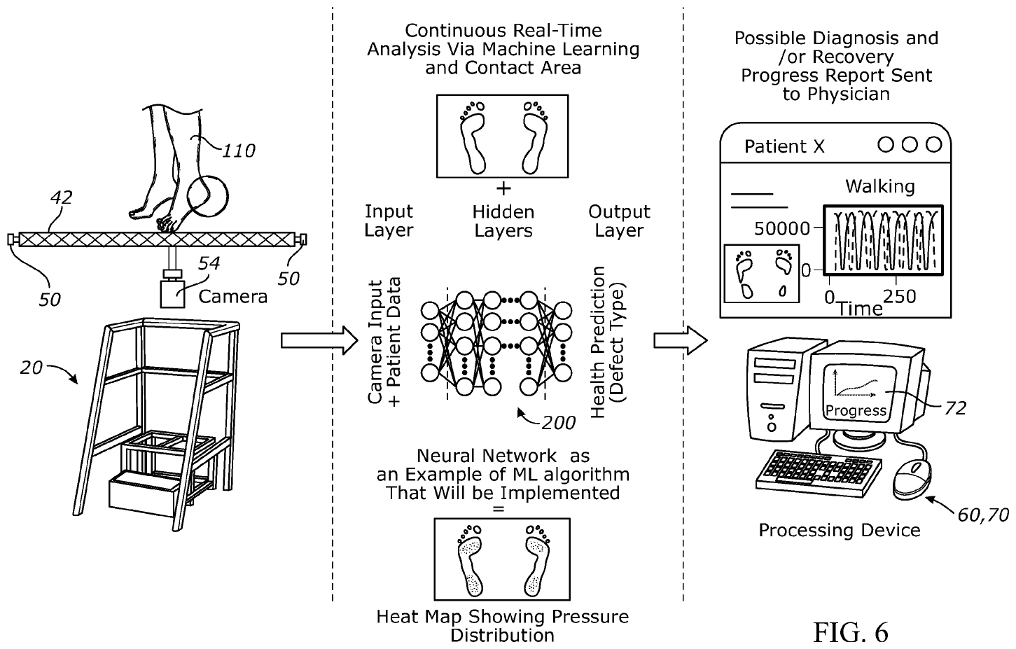

FIGS. 6-8 illustrate an example system and method of generating a real-time pressure distribution map of an subject's feet 110 using an already trained neural network 200. In FIG. 6, the subject 100 stood atop the sensor interface 30 and cyclically raised and lowered their foot 110. More specifically, the subject 100 cyclically loaded and unloaded each foot while standing on the contact surface 42. The multiple wavelength light emitted by the light source 50 was reflected by the loaded/unloaded foot 110 towards the camera 54.

The camera 54 captured multiple images over time of the reflected light and sent those images to the computing device 70 to generate a light intensity map. Individual factors extracted from the images were fed as inputs I to the neural network 200 in order to generate a heat map showing the pressure distribution in real time. The output generator 160 then relied on the pressure distribution map and existing subject data to generate a possible diagnosis and/or recovery progress report that was sent to the attending medical professional. The pressure distribution and/or diagnosis can also be shown in the display 72.

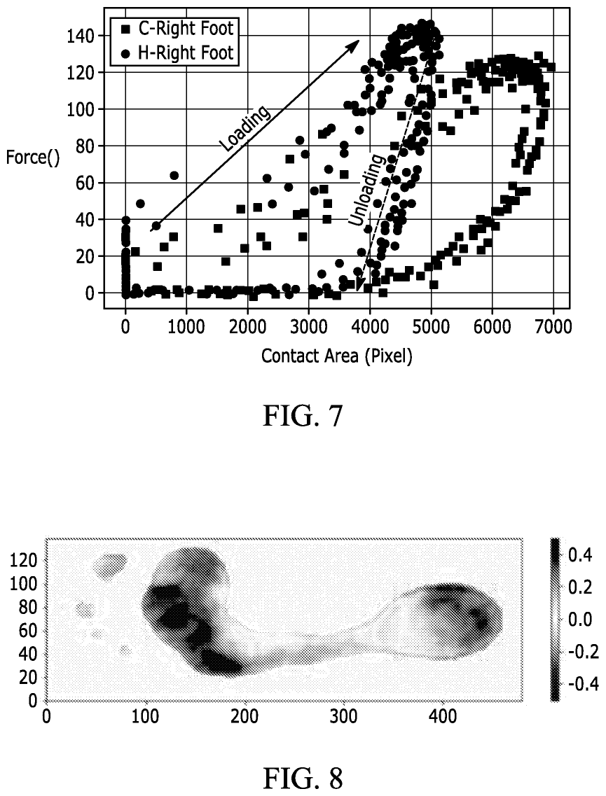

At the same time the computing device 70 analyzed movement of the patient's feet 110. More specifically, FIG. 7 shows the force vs. contact areas for loading and unloading of the forefoot (label: “C-Right Foot”) and heel (label: “H-Right Foot”) during stationary walking on the sensor interface 30. The loading initiated when the foot 110 touched the contact surface 42 (and pressure increases) and continued until the individual 100 started to decrease the force on the foot. The unloading continued until the contact force became zero.

We considered this effect by defining two phases in the contact and considered two different contact models for pressure measurement. At this point (as FIG. 7 indicates) the contact area was not zero. Consequently, it meant that there was some temporary permanent deformations happening to the foot 110 that had not been previously known/addressed. That said, an analytical model solely based on transforming light intensity values to force would have resulted in similar force values as in the loading phase, which according to our work caused very large errors.

In the contrary previous works that considered uniform mechanical properties for feet, we observed a significant spatial change of mechanical properties in each foot, which can differ in pressure distribution maps drastically. We observed in our experiments that the coefficient used in Tompkin can change by 70% for heel and forefoot of a same person. In our work addressed this issue via two methods: 1) tracking different sections of the foot using artificial intelligence and using different contact models for each section of the first; and 2) using a blend of light instead of pure red, green or blue light.

That said, we considered this spatial change during the construction of the pressure maps. As noted, instead of a pure (single wavelength) light, we trapped a combination of red, green and blue lights within the sensor interface. As a result, different parts of the foot with different mechanical properties reflected lights with different properties. We analyzed these differences and created a mechanical properties map (FIG. 8) that could then be used for the construction of a more accurate pressure distribution map via analytical methods. In particular, FIG. 8 reflects the difference between red and blue values at each pixel over the contact area. The map illustrates the difference in mechanical properties between the heel and calluses in the forefoot, namely, darker red regions in comparison to soft areas shown in white to blue colors.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A system for analyzing a body part of a subject, comprising:
a sensor interface having a surface for receiving the body part;
a light source for emitting light having multiple wavelengths within the sensor interface to be reflected by the body part interacting with the surface;
an imaging system for capturing images of the reflected light; and
a computing device for generating a pressure distribution map of the body part on the surface based on the images.

2. The system recited in claim 1, wherein the light source emits a combination of red, blue and green light.

3. The system recited in claim 1, further comprising force sensors for measuring force applied by the body part to the surface.

4. The system recited in claim 3, wherein the computing device generates the pressure distribution map based on the images and the measured force.

5. The system recited in claim 1, wherein the computing device comprises a trained machine learning algorithm to generate the pressure distribution map based on at least one factor extracted from at least one of the images.

6. The system recited in claim 1, wherein the pressure distribution map plots force versus contact area for the body part across the surface.

7. The system recited in claim 1, wherein the surface is planar.

8. The system recited in claim 1, wherein the surface is spherical.

9. The system recited in claim 1, wherein the surface is cylindrical.

10. The system recited in claim 1, wherein the surface is curved.

11. The system recited in claim 1, wherein the computing device is configured to generate a spatial mechanical properties map of the body part based on the images.

12. The system recited in claim 1, wherein the images comprise a distribution of light intensity and different light colors.

13. A method for analyzing a body part of a subject, comprising:
receiving the body part on a contact surface of a sensing interface;
emitting light having multiple wavelengths within the sensing interface;
acquiring images of the light reflected by the body part interacting with the contact surface; and
generating a pressure distribution map of the body part on the contact surface based on the images.

14. The method recited in claim 13, further comprising:
extracting at least one factor from at least one of the images; and
inputting the at least one factor into a trained machine learning algorithm to generate the pressure distribution map.

15. The method recited in claim 13 further comprising:
three-dimensionally analyzing the body part as a viscoelastic material based on the images; and
developing an analytical model based on the three-dimensional analysis; and
generating the pressure distribution map based on the analytical model.

16. The method recited in claim 13, further comprising generating a spatial mechanical properties map of the body part based on the images.

17. The method recited in claim 16, further comprising generating a perfusion map assessing blood circulation in the body part based on the spatial mechanical properties map.

18. The method recited in claim 13, further comprising assessing blood circulation in the body part based on a distribution of different colored light in the reflected light images.

19. The method recited in claim 18, further comprising:
varying the temperature of the body part; and
assessing the blood circulation of the body part at the different temperatures based on changes in the colored light distribution in the light images.

20. The method recited in claim 13, wherein a first pressure distribution map is generated before a treatment of the body part begins and a second pressure distribution map is generated after the treatment of the body part begins.

21. The method recited in claim 13, wherein the contact surface is planar.

22. The method recited in claim 13, wherein the contact surface is spherical.

23. The method recited in claim 13, wherein the contact surface is cylindrical.

24. The method recited in claim 13, wherein the contact surface is curved.
